(19) United States
(12) Patent Application Publication
(10) Pub. No.: US 2025/0332093 A1
(43) Pub. Date: Oct. 30, 2025

(54) DEGRADABLE INTRAUTERINE SYSTEM FOR THE PROLONGED RELEASE OF AN ACTIVE INGREDIENT IN THE UTERINE CAVITY

(71) Applicants: WOMED, MONTPELLIER (FR); CENTRE NATIONAL DE LA RECHERCHE SCIENTIFIQUE, PARIS (FR); UNIVERSITÉ DE MONTPELLIER, MONTPELLIER (FR); ECOLE NATIONALE SUPERIEURE DE CHIMIE DE MONTPELLIER, MONTPELLIER CEDEX 5 (FR)

(72) Inventors: XAVIER GARRIC, MONTPELLIER (FR); GONZAGUE ISSENMANN, PARIS (FR); SALOMÉ LEPRINCE, SAINT LAURENT DE LA SALANQUE (FR)

(21) Appl. No.: 18/698,796

(22) PCT Filed: Oct. 4, 2022
(86) PCT No.: PCT/EP2022/077597

§ 371 Date: Apr. 5, 2024

(30) Foreign Application Priority Data
Oct. 5, 2021 (FR) FR2110538

(51) Int. Cl. A61K 9/00; A61K 47/10; A61K 47/34

(52) U.S. Cl. CPC A61K 9/0039; A61K 47/10; A61K 47/34

(57) ABSTRACT

The present invention relates to a degradable intrauterine system for the prolonged release of an active ingredient in the uterine cavity comprising (a) a degradable A and B block copolymer, wherein the A block is a polyester, the B block is a poly (oxyethylene) (PEO) with a weight-average molecular weight of greater than or equal to 50 kDa; and the ethylene oxide unit/ester unit molar ratio is between 0.05 and 5; (b) at least one polyester homopolymer; and (c) at least one active ingredient intended to be released in the uterine cavity. The invention also relates to a kit comprising at least one intrauterine system according to the invention and means for inserting the system into the uterine cavity.

### TECHNICAL FIELD

The invention relates to a novel degradable intrauterine system for the prolonged release of an active ingredient in the uterine cavity.

### TECHNOLOGICAL BACKGROUND

Pelvic pain is pain in the area of the pelvis. Pelvic pain from the female reproductive system is generally regulated by physiological changes as part of the female menstrual cycle. Dysmenorrhoea, also known as painful periods or menstrual cramps, is the most common type of pelvic pain. Dysmenorrhoea is pain occurring before or at the time of menstruation. This pain is usually intense and can be in the form of pulsating or dull cramps, or constant.

Dysmenorrhoea may be primary (i.e. without an associated underlying cause) or secondary (i.e. due to pelvic anomalies). The symptoms of primary dysmenorrhoea cannot be explained by gynaecological structural pathologies, the pain is attributed to uterine contractions and to uterine ischemia. The symptoms of secondary dysmenorrhoea are due to pelvic anomalies. Virtually any anomaly or any process capable of affecting pelvic viscera may cause secondary dysmenorrhoea. Common causes of secondary dysmenorrhoea include endometriosis (most common cause), uterine adenomyosis and fibroids. Less common causes include congenital malformations (bicornuate uterus, septate uterus, transverse vaginal septum), ovarian cysts and tumours, pelvic inflammatory disease, pelvic congestion, intrauterine adhesions, psychogenic pain, and intrauterine devices (IUDs). (“Dysmenorrhoea”, JoAnn V. Pinkerton, MD, University of Virginia Health System, December 2020).

To date, one of the main treatments for treating dysmenorrhoea consists of the administration of a non-steroidal anti-inflammatory drugs (NSAIDs) which relieve the pain and inhibit prostaglandins. The administration of NSAIDs is generally carried out orally for several days. However, the effectiveness of this treatment is not guaranteed and other hormonal treatments such as danazol, progestins (e.g. levonorgestrel, etonogestrel, depot medroxyprogesterone acetate), gonadotropin-releasing hormone agonists or a levonorgestrel-releasing IUD, may reduce the symptoms of dysmenorrhoea.

For a significant number of patients, the existing treatments provide insufficient relief of the symptoms and notably the pain. Moreover, as the medications are administered orally and not locally, the doses administered are generally high and cause undesirable side effects, such as digestive side effects of varying seriousness (nausea, stomach pain or heartburn, ulcers or gastrointestinal bleeding). They can be responsible for headaches, allergic reactions (skin eruption, asthma) and renal failure in certain rare circumstances.

There is therefore an ongoing need for alternative therapies that better relieve the symptoms of dysmenorrhoea, and notably the pain, while protecting the uterus of the patient, by not being invasive and causing fewer undesirable side effects.

As an example of alternative therapies, there are intrauterine systems which release active compounds such as hormones and notably levonorgestrel. However, these systems are permanent implants, enabling a release of around five years, which are rigid and require the intervention of a health professional to remove them.

### SUMMARY OF THE INVENTION

In this context, the inventors have developed an intrauterine system that meets these needs, and notably an intrauterine system that can be easily inserted into the uterine cavity, that unfolds by itself in the cavity by swelling without being expelled, that degrades in a controlled manner in order to enable the natural elimination thereof through the uterine cervix, and that enables the prolonged release of an active ingredient in the region of the uterine wall for several days or months.

In particular, the inventors have discovered that the use of copolymers based on blocks of polyesters, such as polylactic acid (PLA) or polycaprolactone (PCL), and on blocks of poly (oxyethylène) (PEO), in combination with a polyester homopolymer, makes it possible to produce a material that combines properties of swelling and resorption that are particularly suitable for use in a uterine cavity for a prolonged time and then for the natural elimination thereof through the uterine cervix.

The inventors thus developed a degradable intrauterine system from such a material further comprising an active ingredient, which in “dry” form has dimensions that allow easy insertion from the uterine cervix, and which once in the uterine cavity absorbs the uterine fluids, unfolds in the uterine cavity and releases the active ingredient directly onto or close to the uterine wall. The release of the active ingredient directly in the uterine cavity makes it possible to obtain a local treatment that requires a lower amount of active ingredient compared to medications administered orally or systemically, and therefore makes it possible to reduce the risks of undesirable side effects.

Moreover, the material according to the invention enables a prolonged release of the active ingredient. Specifically, the intrauterine system according to the invention, after having been introduced into the uterine cavity, makes it possible to release an active ingredient in the uterine cavity over a period advantageously of between 10 days and 12 months. Moreover, the system according to the invention, after unfolding and swelling in the uterine cavity, has dimensions that prevent the elimination thereof through the uterine cervix for a period advantageously of between 10 days and 12 months.

In particular, the material according to the invention enables a prolonged release of the active ingredient in the uterine cavity with a burst effect over the 1st day after administration of the system and then a continuous release advantageously during 10 days and 12 months. Such a release profile allows for example to effectively relieve the pain just after the administration and then to maintain the analgesic and anti-inflammatory effect in the following days.

Furthermore, the time for disintegration and evacuation of the intrauterine system according to the invention in/from the uterine cavity is generally advantageously between 10 days and 12 months, which makes it possible to guarantee a sufficient residence time of the intrauterine system in the uterine cavity in order to release the desired sufficient amount of active ingredient over the desired period.

One object of the invention is therefore a degradable intrauterine system for the prolonged release of an active ingredient in the uterine cavity comprising:
(a) a degradable A and B block copolymer, wherein:
   the A block is a polyester;
   the B block is a poly (oxyethylene) (PEO) with a weight-average molecular weight of greater than or equal to 50 kDa; and
   the ethylene oxide unit/ester unit molar ratio is between 0.05 and 5;
(b) at least one polyester homopolymer; and
(c) at least one active ingredient intended to be released in the uterine cavity.

The invention also relates to a kit comprising at least one intrauterine system according to the invention, and means for inserting the system in the uterine cavity.

### BRIEF DESCRIPTION OF THE FIGURES

FIG. 1 is a schematic representation of a degradable intrauterine system according to the invention in the form of a trapezoidal film, particularly suitable for use in a human uterine cavity.

FIG. 2 is a schematic representation, in longitudinal cross-section, of an exemplary embodiment of the kit according to the invention comprising means for inserting a degradable intrauterine system according to the invention in the uterine cavity.

FIG. 3 is a representation of the in vitro release of flurbiprofen over time from a degradable intrauterine system according to the invention.

FIGS. 4a and 4b represent photos of a degradable intrauterine system according to the invention in film form after 24 h of degradation (FIG. 4.a) and 15 days of degradation (FIG. 4.b) under in vitro degradation conditions.

FIG. 5 represents the evolution of the cumulative percentage of flurbiprofen released in vitro over time from ABA 60/40 PCL and ABA 40/60 PCL films composed of the triblock ABA mixture and the homopolymer PCL, and ABA 100/0 PCL films composed of the triblock ABA alone.

FIG. 6 represents the evolution of the cumulative percentage of flurbiprofen released in vitro over time from ABA 30/70 PCL/PLA50 and ABA 20/80 PCL/PLA50 films, composed of the triblock mixture ABA and the homopolymer PCL and PLA50.

FIG. 7 represents the evolution of the cumulative percentage of flurbiprofen released in vitro over time from the 1,000 μm and 2,000 μm thick ABA 6/94 PCL/PLA50 30 30% F films, composed of the triblock mixture ABA and the homopolymer PCL and PLA50.

FIG. 8 represents the evolution of the cumulative percentage of flurbiprofen released in vitro over time from the 1 mm thick films, ABA 20/80 PCL/PLA50+20% F and ABA 6/94 PCL/PLA50+30% F, composed of the triblock mixture ABA and the homopolymer PCL and PLA50.

FIG. 9 represents the evolution of the mass loss of ABA 20/80 PCL/PLA50+20% F films at different times (at 11 H, 25 H, day 15, day 28 and day 56) after being implanted into rat uterine horns.

FIG. 10 represents the amount of flurbiprofen absorbed in rat uterine tissue after implantation of “ABA 6/94 PCL/PLA50+30% F” film (at 11 H, 25 H, day 15, day 28 and day 56) and the amount of flurbiprofen absorbed in uterine tissue after oral administration of flurbiprofen (at at 11 H, 2 5H, and day 15).

### DETAILED DESCRIPTION

The inventors have developed a degradable intrauterine system for the prolonged release of an active ingredient in the uterine cavity which system has mechanical and chemical properties particularly suitable for use in the medical field, and in particular for the treatment of pelvic pain and/or gynaecological disorders in female mammals, notably in women. Specifically, the swelling and unfolding properties of the polymer composition used to form the intrauterine system, combined with the active principle, mean that it is possible to use it in the uterine cavity to reliably treat, in a prolonged manner, gynaecological disorders in women, such as dysmenorrhoea.

### The Degradable Intrauterine System

One object of the invention is a degradable intrauterine system for the prolonged release of an active ingredient in the uterine cavity comprising:
(a) a degradable A and B block copolymer, wherein:
   the A block is a polyester;
   the B block is a poly (oxyethylene) (PEO) with a weight-average molecular weight of greater than or equal to 50 kDa; and
   the ethylene oxide unit/ester unit molar ratio is between 0.05 and 5;
(b) at least one polyester homopolymer; and
(c) at least one active ingredient intended to be released in the uterine cavity.

In the context of the invention, the expression “between x and y” means that the values x and y are included.

According to the invention, the term “polyester” denotes any polymer wherein the repeat units of the main chain contain the ester function and which can be used in the medical field. Notably, polyesters is understood to mean aliphatic polyesters such as poly(lactic acid) (PLA), poly(glycolic acid) (PGA), polycaprolactone (PCL), poly(lactic-co-glycolic acid) (PLGA), polybutyrolactone (PBL), polyhydroxyalkanoates (PHA), and copolymers thereof.

In one preferred embodiment, the polyester (A block) is chosen from poly (lactic acid) (PLA), poly(glycolic acid) (PGA), polycaprolactone (PCL) and copolymers thereof. Preferably, the polyester of the A block is chosen from PLA and PCL.

Preferentially, the polyester is in a non-crosslinked form.

The poly(lactic acid) may be poly(L-lactic acid), poly(D-lactic acid) or poly(D,L-lactic acid). Advantageously, use is made of poly(D,L-lactic acid) (PDLLA). In this case, the polymer preferentially comprises at least 50 mol % of L-lactic acid, and may particularly comprise at least 60%, 70%, 75%, 80%, 85%, 90%, 95% or 99% of L-lactic acid. Specifically, by modifying the percentage of L-lactic acid relative to the D-lactic acid, it is possible to modify the rate of degradation of the A and B block copolymer. An increase in the level of L-lactic acid makes it possible to slow down the degradation rate of the copolymer. In certain embodiments of the invention, the composition comprises 100% of PLLA as A blocks.

In the context of the invention, the poly(oxyethylene) (PEO) is typically a linear polyether produced from ethylene oxide or ethylene glycol monomers, preferably ethylene oxide monomers. Thus, according to the invention, the B block may also be a polyethylene glycol (PEG) having a high molecular weight greater than or equal to 50 kDa, notably having a molecular weight as defined below.

According to the invention, the poly(oxyethylene) (PEO) used for the B block has a high molecular weight, so that the total molecular weight of the PEO in the copolymer is greater than or equal to 50 kDa. In the context of the invention, the terms “molecular mass” and “molecular weight” are used equally to denote, unless otherwise mentioned, the weight-average molecular weight (Mw). According to the invention, the Mw is determined by size exclusion chromatography carried out in dimethylformamide as analytical solvent, using a standard range of poly(ethylene glycol).

Advantageously, the total molecular weight of the PEO in the A and B block copolymer is between 50 kDa and 300 kDa. For example, the PEO blocks have a molecular weight of 50 kDa, 75 kDa, 80 kDa, 85 kDa, 90 kDa, 95 kDa, 100 kDa, 105 kDa, 110 kDa, 115 kDa, 120 kDa, 125 kDa, 150 kDa, 200 kDa, 225 kDa, 250 kDa, 275 kDa or 300 kDa. In one particular embodiment, the PEO blocks used have a molecular weight of between 75 kDa and 150 kDa, preferentially between 80 kDa and 125 kDa, more preferentially between 90 kDa and 115 kDa, more preferably between 90 kDa and 110 kDa. In one particular embodiment, the PEO blocks used have a molecular weight of between 95 kDa and 105 kDa.

According to the invention, the PEO block used in the A and B block copolymer advantageously has an inherent viscosity of between 0.04 mg/ml and 0.6 mg/ml, preferentially between 0.08 mg/ml and 0.5 mg/ml, and more preferably between 0.1 mg/ml and 0.3 mg/ml when it is measured by an Ubbelohde type capillary viscometer at a concentration of 1 g/l, at 25° C. in chloroform.

Advantageously, the A and B block copolymer is chosen from AB diblock copolymers, or ABA or BAB triblock copolymers, or mixtures thereof, notably [ABA and BAB], [AB and ABA], [AB and BAB], [ABA and BAB and AB]. In one preferred embodiment, the A and B block copolymers are selected from ABA or BAB triblock copolymers, and preferentially ABA triblock copolymers.

According to the invention, in an AB and/or ABA copolymer, each PEO block (B block) has a molecular weight greater than or equal to 50 kDa and advantageously between 50 kDa and 300 kDa, preferentially between 75 kDa and 150 kDa, preferably between 80 kDa and 125 kDa, more preferentially between 90 kDa and 115 kDa, more preferably between 90 kDa and 110 kDa, or even between 95 kDa and 105 kDa; whilst in a BAB copolymer, the sum of the molecular weights of the PEO blocks in said copolymer is greater than or equal to 50 kDa and advantageously between 50 kDa and 300 kDa, preferentially between 75 kDa and 150 kDa, preferably between 80 kDa and 125 kDa, more preferentially between 90 kDa and 115 kDa, more preferably between 90 kDa and 110 kDa, or even between 95 kDa and 105 kDa.

In the context of the invention, the ethylene oxide unit/ester unit molar ratio in the copolymer (a), also referred to as the EO/LA ratio in the present description, represents the molar ratio of each of the repeat units of the A and B blocks. The B block being PEO, the repeat units are ethylene oxides (“ethylene oxide unit” or EO), whilst the repeat units of the A block (“ester unit”) are carboxylic acids such as lactic acid units. According to the invention, the EO/LA ratio in the A and B block copolymer is between 0.05 and 5, advantageously between 0.1 and 4, and preferably between 0.1 and 3. The EO/LA ratio is measured from the proton NMR (nuclear magnetic resonance) spectrum in deuterated chloroform of the copolymer wherein it is possible to identify the chemical shifts of the characteristic peaks of the PLA-PEO-PLA copolymers: CH (PLA): 5.1 ppm; CH₂(PEO): 3.5 ppm; CH₃(PLA): 1.5 ppm). According to the invention, controlling the EO/LA ratio makes it possible to control the swelling and unfolding properties of the intrauterine system, and also the degradation time. Typically, the lower the EO/LA ratio, the longer the degradation time.

According to the invention, an “aqueous medium” refers to a medium having an osmolarity similar to the osmolarity of biological fluids. Use is commonly made, as aqueous medium, of phosphate-buffered saline (PBS) considered to be representative of biological fluids.

According to the invention, a “humid medium” refers to a medium equivalent to the aqueous medium, i.e. a medium having an osmolarity similar to the osmolarity of biological fluids, but the humid medium is not liquid. The uterine cavity can be characterized as a non-liquid humid medium.

In one particular embodiment, the system according to the invention comprises ABA triblock copolymers, wherein the A block is PDLLA or PCL, and the B block is PEO having a molecular weight of between 90 kDa and 110 kDa, wherein the EO/LA molar ratio is between 0.05 and 5, preferably between 0.1 and 3.

In one particular embodiment, the system according to the invention comprises ABA triblock copolymers, where the A block is PDLLA comprising between 50% and 100% L-lactic acid (PLA50-PLA100) and the B block is PEO having a molecular weight of between 90 kDa and 110 kDa, wherein the EO/LA molar ratio is between 0.05 and 5, preferably between 0.1 and 3.

In one particular embodiment, the system according to the invention comprises ABA triblock copolymers, where the A block is PDLLA comprising between 80% and 100% L-lactic acid (PLA80-PLA100) and the B block is PEO having a molecular weight of between 90 kDa and 110 kDa, wherein the EO/LA molar ratio is between 0.05 and 5, preferably between 0.1 and 3.

In one particular embodiment, the system according to the invention comprises ABA triblock copolymers, where the A block is PDLLA comprising at least 90% L-lactic acid and the B block is PEO having a molecular weight of between 90 kDa and 110 kDa, wherein the EO/LA molar ratio is between 0.05 and 5, preferably between 0.1 and 3.

In one particular embodiment, the system according to the invention comprises ABA triblock copolymers, where the A block is polycaprolactone (PCL) and the B block is PEO having a molecular weight of between 90 kDa and 110 kDa, wherein the EO/LA molar ratio is between 0.05 and 5, preferably between 0.1 and 3.

The A and B block copolymer according to the invention can be obtained by any block copolymer synthesis method known to the person skilled in the art. For example, an ABA type copolymer may be obtained by chain polymerization from the ends of the B block. Typically, a ring-opening polymerization initiated by the terminal hydroxyls of the PEO block in the presence of a catalyst such as tin octanoate is carried out. This polymerization can be carried out in the absence or presence of solvents. A BAB type copolymer may for example be prepared by coupling of methoxy-PEO to a polyester chain, the two chain ends of which are carboxylic acid functions. Such a “difunctionalized” polyester is obtained for example by treating a polyester chain with succinic or adipic anhydride.

In the context of the invention, the degradable intrauterine system also comprises at least one polyester homopolymer and notably at least one degradable polyester homopolymer. The addition of at least one polyester homopolymer to the system according to the invention makes it possible to improve the release and degradability properties of the system and notably to extend the release time of the active ingredient and delay the degradation and elimination of the system through the cervix. Advantageously, the polyester homopolymer (b) is chosen from poly (lactic acid) (PLA), poly(glycolic acid) (PGA), poly(lactic-co-glycolic acid) (PLGA), polycaprolactone (PCL), polybutyrolactone (PBL), and polyhydroxyalkanoates (PHA), advantageously from poly(lactic acid) (PLA) and polycaprolactone (PCL). In one particular embodiment, the degradable intrauterine system comprises one or two polyester homopolymer(s) chosen from the above-mentioned list. For example, the degradable intrauterine system may comprise PLA and/or PCL as homopolymer(s) (b).

In one advantageous embodiment, the molar mass of the polyester homopolymer (b) is chosen so as to obtain the release and degradability profile desired for the intrauterine system according to the invention. For example, the higher the molar mass of the polyester homopolymer (b), the longer the time period before degradation and elimination of the system according to the invention through the uterine cervix and the more prolonged the release. Advantageously, the polyester homopolymer (b) has a number-average molar mass of between 25 000 g/mol and 150 000 g/mol, preferably between 50 000 g/mol and 125 000 g/mol, notably between 50 000 g/mol and 100 000 g/mol, in particular between 80 000 and 100 000 g/mol. The number-average molar mass of the polyester homopolymer (b) can be measured by size exclusion chromatography performed in tetrahydrofuran (THF) or dimethylformamide (DMF) as analytical solvent, with polystyrene or polymethyl methacrylate (PMMA) standards.

In one particular embodiment, the polyester homopolymer (b) is a poly (lactic acid) (PLA) homopolymer with a number-average molar mass of between 25 000 g/mol and 150 000 g/mol, preferably between 50 000 g/mol and 125 000 g/mol.

In another particular embodiment, the polyester homopolymer (b) is a polycaprolactone (PCL) homopolymer with a number-average molar mass of between 25 000 g/mol and 150 000 g/mol, preferably between 50 000 g/mol and 125 000 g/mol.

In another particular embodiment, the polyester homopolymer (b) is a blend of polycaprolactone (PCL) homopolymer and poly (lactic acid) (PLA) homopolymer, said homopolymers having a number-average molar mass of between 25 000 g/mol and 150 000 g/mol, preferably between 50 000 g/mol and 125 000 g/mol.

The polyester homopolymer (b) according to the invention is prepared according to methods known to the person skilled in the art, for example by polycondensation or by ring-opening polymerization methods in the presence of a catalyst. For example, the PCL may be prepared by ring-opening polymerization of &-caprolactone with use of a catalyst. Likewise, the PLA may be prepared by polycondensation or by ring-opening polymerization of lactide in the presence of a catalyst.

In the context of the invention, the A and B block copolymer (a) and the homopolymer (b) coexist within the system according to the invention but do not react together or do not crosslink together. The mixing of the A and B block copolymer (a) and of the homopolymer(s) (b) may be carried out by any means known to the person skilled in the art, for example by solubilization of powders of copolymers and homopolymers in a common solvent (dichloromethane for example) followed by a step of solvent evaporation, or by cold or hot mixing (temperature between 30° C. and 190° C.) of powders of homopolymers and copolymers.

In the context of the invention, the copolymer (a)/homopolymer (b) weight ratio is advantageously between 99/1 and 1/99. In particular, the copolymer (a)/homopolymer (b) weight ratio is advantageously between 98/2 and 2/98, more particularly between 97/3 and 3/97, more particularly between 96/4 and 4/96. In the context of the invention, the copolymer (a)/homopolymer (b) weight ratio is advantageously between 95/5 and 1/99. In the context of the invention, the copolymer (a)/homopolymer (b) weight ratio is advantageously between 95/5 and 5/95. According to the invention, the copolymer (a)/homopolymer (b) weight ratio is also chosen so as to obtain the release and degradability profile desired for the intrauterine system according to the invention. For example, the lower this weight ratio, the longer the time period before degradation and elimination of the system according to the invention through the uterine cervix. Thus, in order to obtain a prolonged release system over a short period (i.e. between 10 days and 30 days approximately), the copolymer (a)/homopolymer (b) weight ratio is advantageously between 95/5 and 50/50, in particular between 90/10 and 50/50, in particular between 80/20 and 50/50. In this embodiment, the copolymer (a)/homopolymer (b) weight ratio may be for example 95/5, 90/10, 85/15, 80/20, 75/25, 70/30, 65/35, 60/40, 55/45 or 50/50. In order to obtain a prolonged release system over a short period (i.e. between 10 days and 30 days approximately), the copolymer (a)/homopolymer (b) weight ratio is advantageously between 99/1 and 50/50, in particular between 98/2 and 50/50, in particular between 97/3 and 50/50, in particular between 96/4 and 50/50. In this embodiment, the copolymer (a)/homopolymer (b) weight ratio may be for example 96/4, 97/3, 98/2 or 99/1.

Similarly, in order to obtain a prolonged release system over a longer period (i.e. between 30 days and 12 months approximately), the copolymer (a)/homopolymer (b) weight ratio is advantageously between 50/50 and 5/95, in particular between 50/50 and 10/90, in particular between 50/50 and 20/80. In this embodiment, the copolymer (a)/homopolymer (b) weight ratio may be for example 50/50, 45/55, 40/60, 35/65, 30/70, 25/75, 20/80, 15/85, 10/90 or 5/95. In order to obtain a prolonged release system over a longer period (i.e. between 30 days and 12 months approximately), the copolymer (a)/homopolymer (b) weight ratio can also be advantageously between 50/50 and 1/99, in particular between 50/50 and 2/98, in particular between 50/50 and 3/97, in particular between 50/50 and 4/96. In this embodiment, the copolymer (a)/homopolymer (b) weight ratio may be for example 4/96, 3/97, 2/98 or 1/99.

In one particular embodiment, the system according to the invention comprises:
a) an ABA triblock copolymer, where the A block is PDLLA, and the B block is PEO having a molecular weight of between 90 kDa and 110 kDa, wherein the EO/LA molar ratio is between 0.05 and 5, preferably between 0.1 and 3;
b) a PLA homopolymer and/or a PCL homopolymer; and
c) at least one active ingredient intended to be released in the uterine cavity, wherein the copolymer (a)/homopolymer (b) weight ratio is advantageously between 50/50 and 1/99.

In one particular embodiment, the system according to the invention comprises:
a) an ABA triblock copolymer, where the A block is PDLLA, and the B block is PEO having a molecular weight of between 90 kDa and 110 kDa, wherein the EO/LA molar ratio is between 0.05 and 5, preferably between 0.1 and 3;
b) a PLA homopolymer and/or a PCL homopolymer; and
c) at least one active ingredient intended to be released in the uterine cavity, wherein the copolymer (a)/homopolymer (b) weight ratio is advantageously between 50/50 and 5/95.

In one particular embodiment, the system according to the invention comprises:
a) an ABA triblock copolymer, where the A block is PDLLA, and the B block is PEO having a molecular weight of between 90 kDa and 110 kDa, wherein the EO/LA molar ratio is between 0.05 and 5, preferably between 0.1 and 3;
b) a PLA homopolymer and/or a PCL homopolymer; and
c) at least one active ingredient intended to be released in the uterine cavity, wherein the copolymer (a)/homopolymer (b) weight ratio is advantageously between 95/5 and 50/50.

In one particular embodiment, the system according to the invention comprises:
a) an ABA triblock copolymer, where the A block is PCL, and the B block is PEO having a molecular weight of between 90 kDa and 110 kDa, wherein the EO/LA molar ratio is between 0.05 and 5, preferably between 0.1 and 3;
b) a PLA homopolymer and/or a PCL homopolymer; and
c) at least one active ingredient intended to be released in the uterine cavity, wherein the copolymer (a)/homopolymer (b) weight ratio is advantageously between 50/50 and 5/95.

In one particular embodiment, the system according to the invention comprises:
a) an ABA triblock copolymer, where the A block is PCL, and the B block is PEO having a molecular weight of between 90 kDa and 110 kDa, wherein the EO/LA molar ratio is between 0.05 and 5, preferably between 0.1 and 3;
b) a PLA homopolymer and/or a PCL homopolymer; and
c) at least one active ingredient intended to be released in the uterine cavity, wherein the copolymer (a)/homopolymer (b) weight ratio is advantageously between 95/5 and 50/50.

In the context of the invention, the intrauterine system for prolonged release of an active ingredient in the uterine cavity comprises the A and B block copolymer, the polyester homopolymer as described above, and an active ingredient, said active ingredient being intended to be released in the uterine cavity.

In one preferred embodiment, in the system according to the invention, the active ingredient is not covalently bound to the copolymer a) or to the homopolymer b).

In the context of the invention, the active ingredient intended to be released in the uterine cavity is advantageously an active ingredient for treating or preventing pelvic pain (such as dysmenorrhoea, endometriosis, or adenomyosis), or a gynaecological disorder (such as fibroids, endometrial cancer, poor endometrial receptivity, thin endometrium, infections, haemorrhage (menorrhagia, metrorrhagia), cancer side effects, ageing, menopause, or vaginal dryness), in female mammals and notably in women. More advantageously, the active ingredient is chosen from anti-infectives, such as antibiotics, antifungals or antivirals; steroidal or non-steroidal anti-inflammatory drugs; vasoconstrictors; vasodilators; uterine relaxants; oxytocics; hormones, hormone analogues, hormone agonists, and hormone antagonists; and anti-cancer drugs. In one particular embodiment, the system according to the invention may comprise a combination of at least two active ingredients mentioned above. Preferentially, the active ingredient used in the system according to the invention is capable of diffusing to the outside of the system when it is in an aqueous or humid medium.

Examples of anti-infective active ingredients that may be chosen within the context of the invention include: chlorhexidine, doxycycline, azithromycin, clindamycin, gentamicin, ampicillin, amoxicillin, metronidazole, nystatin, miconazole, cidofovir, and imiquimod.

Examples of non-steroidal anti-inflammatory (NSAID) active ingredients that may be chosen within the context of the invention include: aminoarylcarboxylic acid derivatives such as enfenamic acid, etofenamate, flufenamic acid, isonixin, meclofenamic acid, mefenamic acid, niflumic acid, talniflumate, terofenamate and tolfenamic acid; arylacetic acid derivatives such as acemetacin, aceclofenac, amfenac, bufexamac, cinmetacin, clopirac, diclofenac sodium, etodolac, felbinac, fenclofenac, fenclorac, fenclozic acid, fentiazac, glucametacin, ibufenac, indomethacin, isofenacine, isofezolacine, proglumetacin, sulindac, tiaramide, tolmetin and zomepirac; arylbutyric acid derivatives such as bumadizone, butibufen, fenbufen and xenbucin; arylcarboxylic acids such as clidanac, ketorolac and tinoridine; arylpropionic acid derivatives such as alminoprofen, benoxaprofen, bucloxic acid, carprofen, fenoprofen, flunoxaprofen, flurbiprofen, ibuprofen, ibuproxam, indoprofen, ketoprofen, loxoprofen, miroprofen, naproxen; pyrazoles such as difenamizole and epirizole; pyrazolones such as apazone, benzpiperylone, feprazone, mofebutazone, morazone, oxyphenbutazone, phenylbutazone, pipebuzone, propyphenazone, ramifenazone, suxibuzone and thiazolinobutazone; salicylic acid derivatives such as acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphtyl salicylate, phenyl acetylsalicylate, phenyl salicylate, salacetamide, salicylamine o-acetic acid, salicylsulfuric acid, salsalate and sulfasalazine; thiazinecarboxamides such as droxicam, isoxicam, meloxicam, piroxicam and tenoxicam; ε-acetamidocaproic acid, s-adenosyl methionine, 3-amino-4-hydroxybutyric acid, amixetrine, bendazac, benzydamine, bucolome, difenpiramide, ditazole, emorfazone, nabumetone, nimesulide, orgotein, oxaceprol, paranyline, proquazone, proxazole, tiaprofenic acid, rofecoxib, celecoxib, parecoxib, and tenidap; hydroxychloroquine; and pharmaceutically acceptable salts and esters thereof; and also combinations thereof.

Examples of steroidal anti-inflammatory (SAID) active ingredients that may be chosen within the context of the invention include: budesonide, triamcinolone, cortivazol, fluticasone, mometasone, prednisolone, methylprednisolone, hydrocortisone, flumethasone pivalate, triamcinolone, dexamethasone, betamethasone, amcinonide and difluprednate.

Examples of vasoconstrictor active ingredients that may be chosen within the context of the invention include: norephedrine, phenylephrine, phenylpropanolamine, phenyltoloxamine, pseudoephedrine, ephedrine, fenoxazoline, naphazoline, oxymetazoline, and tymazoline.

Examples of vasodilator active ingredients that may be chosen within the context of the invention include: sildenafil citrate, glyceryl trinitrate, nitroglycerin, and nifedipine.

Examples of uterine relaxant (anti-contraction or tocolytic) active ingredients that may be chosen within the context of the invention include: atosiban, ritodrine, salbutamol, and terbutaline.

Examples of oxytocic (or uterotonic) active ingredients that may be chosen within the context of the invention include: methylergomatrine, oxytocin, dinoprostone, sulprostone, gemeprost, and misoprostol.

Examples of hormones, hormone analogues, hormone agonists and hormone antagonists that may be chosen within the context of the invention include: estradiol, levonorgestrel, gestodène, drospirenone, norgestimate, GnRH agonists (such as triptorelin, leuprorelin, buserelin, gonadorelin, nafarelin), GnRH antagonists (such as cetrotide, ganirelix), menotropin, urofillitropine, danazol, medroxyprogesterone, norethisterone acetate, dienogest, megestrol, tamoxifen, and ulipristal acetate.

Examples of anti-cancer drugs that may be chosen within the context of the invention include: cisplatin, carboplatin, 5-fluorouracil, mitomycin C, paclitaxel, docetaxel, vinorelbine, gemcitabine, capecitabine, pemetrexed, and topotecan.

In the context of the invention, the active ingredient intended to be released in the uterine cavity can also be a selective progesterone receptor modulator, such as mifepristone, asoprisnil, onapristone and ulipristal acetate.

In one preferred embodiment, the active ingredient used is chosen from non-steroidal anti-inflammatory drugs (NSAIDs) and hormones, hormone analogues, hormone agonists and hormone antagonists, advantageously from NSAIDs. NSAIDs that are particularly preferred within the context of the invention are chosen from ibuprofen, flurbiprofen, diclofenac, ketoprofen, aspirin, naproxen, ketorolac, tiaprofenic acid, mefenamic acid, tenoxicam, piroxicam, meloxicam, rofecoxib, celecoxib, parecoxib, fenoprofen, alminoprofen, hydroxychloroquin and indomethacin, preferably flurbiprofen.

Advantageously, the content of active ingredient in the intrauterine system according to the invention is between 0.01% and 60% by weight, preferably between 1% and 60% by weight, relative to the total weight of the system. In particular, the content of active ingredient in the intrauterine system according to the invention is advantageously between 10% and 50% by weight, preferably between 20% and 50% by weight, in particular between 30% and 50% by weight, relative to the total weight of the system. In one particular embodiment, when the active ingredient is chosen from NSAIDs, the content of active ingredient in the intrauterine system according to the invention is between 1% and 60% by weight, preferably between 10% and 50% by weight, in particular between 20% and 50% by weight, relative to the total weight of the system.

In another particular embodiment, when the active ingredient is chosen from hormones, hormone analogues, hormone agonists and hormone antagonists, the content of active ingredient in the intrauterine system according to the invention is between 0.01% and 60% by weight, preferably between 0.1% and 50% by weight, in particular between 0.5% and 40% by weight, relative to the total weight of the system.

The preparation of the intrauterine system according to the invention can be carried out by any means known to the person skilled in the art, and notably by integration of the desired active ingredient during or after the forming of a polymer matrix comprising the copolymer a) and the homopolymer b).

According to another particular embodiment, the system according to the invention can be prepared by integration of the active ingredient during the forming of the polymer matrix by means known to the person skilled in the art, and notably by one of the following means:
impregnating/swelling of base polymers of the polymer matrix (copolymer a) and homopolymer b)) in a solution containing at least one active ingredient;
mixing dry powders of the base polymers of the polymer matrix (copolymer a) and homopolymer b)) and of active ingredient;
mixing by melting or softening of powders of base polymers of the polymer matrix (copolymer a) and homopolymer b)) and of active ingredient;
mixing of a polymer (copolymer a) and homopolymer b)) solution and of a powder of active ingredient leading to a suspension or a solution; and
mixing of a solution of active ingredient and of a powder of base polymers of the polymer matrix (copolymer a) and homopolymer b)) leading to a suspension or a solution.

The system according to the invention can then be formed by means known to the person skilled in the art, and notably by hot pressing, hot injection, extrusion, solvent evaporation using, for example, dichloromethane, electrospinning, moulding or 3D printing, from the dispersion as obtained in the preceding step, comprising the active ingredient in polymer matrix (copolymer a) and homopolymer b)).

According to one particular embodiment, the forming of the polymer matrix may be carried out by any means known to the person skilled in the art, for example by extrusion, solvent evaporation using for example dichloromethane, hot pressing, hot injection, electrospinning, moulding or 3D printing. The system according to the invention may then be obtained by addition of the active principle within the polymer matrix after forming by one of the following means:
impregnating/swelling of the polymer matrix (copolymer a) and homopolymer b)) in a solution or suspension comprising at least one active ingredient;
coating of the polymer matrix (copolymer a) and homopolymer b)) using a solution or a suspension comprising at least one active ingredient, and optionally a water-soluble excipient, in order to form a coating on the surface of the polymer matrix; or
depositing on the surface of the polymer matrix (copolymer a) and homopolymer b)) a powder comprising an active ingredient and a water-soluble excipient then hot pressing of the polymer matrix and the powder, it being possible for the surface of the polymer matrix to be coated with a solvent, such as acetone, ethanol or dichloromethane, to facilitate the attachment of the powder to the polymer matrix.

Thus, according to the invention, the active ingredient can be integrated into the very structure of the polymer matrix comprising the copolymer a) and the homopolymer b), and/or form an at least partial coating on the outer surface of the polymer matrix comprising the copolymer a) and the homopolymer b) after forming.

Generally, the thickness of the system according to the invention that is obtained depends on the amount of polymer matrix used and on the surface of the support or of the mould used for the forming.

The system according to the invention may take any type of form suitable for the morphology of the uterine cavity. Thus, the system according to the invention may take the form of a film, a tube, a powder, a porous structure, such as a 2D or 3D matrix, a complex 3D structure, a gel or a porous or non-porous hydrogel, etc.

A film is understood to mean a two-dimensional material, resulting for example from the evaporation on a flat surface of the solvent that solubilized the A and B block copolymer a) and the homopolymer b) according to the invention. The thickness of such a film is advantageously between a few microns and several hundred microns, and notably between 10 μm and 1000 μm. In one particular embodiment, the film has a thickness between 200 μm and 600 μm. The thickness is understood to be “dry”, in the sense that it is measured (for example by optical microscopy) under anhydrous conditions, after forming and optionally the complete evaporation of the solvent used to solubilize the copolymer.

The dimensions of the film may be adapted according to the requirements, notably by cutting a film of larger dimensions to the desired dimensions.

The films may be folded to form tubes, or sleeves, held closed if need be by a suture or adhesive bonding, or accordion folded. Tubes may also be obtained directly by forming around a cylinder or by extrusion.

In the context of the invention, a tube denotes a hollow or solid, three-dimensional cylindrical object, the walls of which are formed from a film of A and B block copolymer a) and homopolymer b) according to the invention. Preferentially, the diameter of such a tube is several hundreds of microns, and notably between 500 μm and 5000 μm. In one particular embodiment, the tube has a wall thickness of 1000 μm and a diameter of 3000 μm. For example, the degradable uterine system can have the shape of a cylinder; the diameter can be from 0.1 mm to 9 mm, and preferably between 2 mm and 6 mm. The length of the cylinder can be from 0.5 cm to 20 cm and preferably between 3 cm to 10 cm. The cylinder can be straight or bent, in a U-shape, V-shape or L-shape, in a serpentine shape or wound around a core.

In one particular embodiment, the system according to the invention is obtained by forming on a support intended to form a part of said system. For example, the material is dried on a woven or knitted textile consisting of another polymer, the assembly thus forming a composite material.

Advantageously, the system according to the invention comprises only or consists of the copolymer a), the homopolymer b), at least one active ingredient, and optionally traces of solvent.

In certain cases, the system according to the invention may further comprise an excipient or additive. This excipient or additive may for example be added to the composition based on copolymers before or during the forming of the material, so as to be dispersed in the copolymer a) and the homopolymer b). In other words, it is possible to impregnate or cover the material with this excipient or additive after forming.

The system according to the invention exhibits swelling and unfolding properties that are particularly suitable for intrauterine use. In particular, the specific properties of the intrauterine system according to the invention enable the easy administration thereof in the uterine cavity, then the unfolding thereof by swelling, and the prolonged-release of the active ingredient in the vicinity of the uterine wall. In one particular embodiment, the specific properties of the intrauterine system according to the invention allow contacting of said system with the uterine wall after swelling and unfolding. Such contacting with the uterine wall notable enables a more local and faster treatment.

Advantageously, the material according to the invention has, in an aqueous or humid medium having an osmotic pressure identical to that of biological fluids, a swelling ratio of between 1 and 20, and preferably between 3 and 15. The swelling ratio is measured in the following manner: a strip of dry material is weighed before immersing it for 24 hours at 37° C. in a saline medium (PBS 1X) with stirring. After 24 hours, the excess PBS is removed with absorbent paper and the strip is weighed again. The swelling ratio corresponds to the weight of the strip of humid material/weight of the strip of dry material ratio. The swelling ratio of the material is proportional to its water uptake percentage, which corresponds to the ratio [(weight of the strip of humid material−weight of the strip of dry material)/weight of the strip of dry material]×100.

The swelling of the material is accompanied by an increase in surface area and volume (“hydrated” surface area or volume) which is particularly advantageous in medical use for the local administration of an active ingredient, since this promotes the unfolding of the material in the uterine cavity, in particular to be as close as possible to the uterine wall.

The increase in surface area is rapid and may notably reach 200% to 300% within a few minutes to a few hours in an aqueous or humid medium. In particular, the increase in surface area may reach a maximum of 100% within 30 minutes and/or a maximum of 300% within 24 hours. The increase in surface area is accompanied by an increase in volume of the material, which is measured visually, under the same conditions as during the measurement of the swelling ratio, by modifying only the residence times in the saline solution. The increase in surface area corresponds to the ratio [(surface area of the “hydrated” strip after an immersion time t−surface area of the “dry” strip)/surface area of the “dry” strip]×100.

The specific swelling properties of the intrauterine system according to the invention thus enable it to unfold in the cavity without risk of being expelled and advantageously to come into contact with the uterine wall, in order to release the active ingredient locally.

According to a preferred embodiment, the intrauterine system according to the invention enables a prolonged release of the active ingredient in the uterine cavity over at least 10 days, such as for example over 10 days, 12 days, 15 days, 21 days, 28 days, 30 days, 2 months, 3 months, 4 months, 5 months or 6 months. Advantageously, the prolonged release of the active ingredient is carried out between 10 days and 12 months, in particular between 10 days and 9 months, in particular between 10 days and 6 months, in particular between 10 days and 3 months, in particular between 10 days and 2 months, in particular between 10 days and 30 days. Advantageously, the prolonged release of the active ingredient is a continuous prolonged release. Within the context of the invention, the amounts of active ingredient released at each release time may be measured by HPLC with a UV detector, fluorescence detector or mass spectrometer.

One particularly advantageous additional feature of the system according to the invention is that it is degradable in an aqueous or humid medium. In particular, the system according to the invention degrades after a residence time in an aqueous or humid medium of between 10 days and 12 months, preferentially between 10 days and 9 months, preferentially between 10 days and 6 months, in particular between 10 days and 3 months, in particular between 10 days and 70 days. The degradation time in an aqueous or humid medium is notably measured in vitro on a uterine model. The degradation of the material is due to the progressive hydrolysis of the ester bonds of the polyester blocks followed by a solubilization of the blocks containing PEO. The loss of the mechanical properties of the material is directly linked to its degradation. The degradation may also be evaluated by measuring, over time, the decrease in the molecular weight of a strip of material, after immersion at 37° C. in a saline medium (PBS 1X) with stirring, for example by size exclusion chromatography. The solubilization of the PEO blocks and the hydrolysis of the polyester blocks in the uterine cavity is gradual and controlled to allow the elimination of the material in the desired time. The degradation properties of the system according to the invention thus enable the system to remain intact in the uterine cavity for a time sufficient to release the active ingredient at the uterine wall in a prolonged manner, and then to degrade sufficiently to allow the natural elimination thereof.

Advantageously, the degradable intrauterine system according to the invention is in the form of a film having a triangular or trapezoidal shape, in order to be in contact with the wall of the uterine cavity, once hydrated and unfolded by swelling.

The film may for example have a dry thickness of 300 to 600 microns.

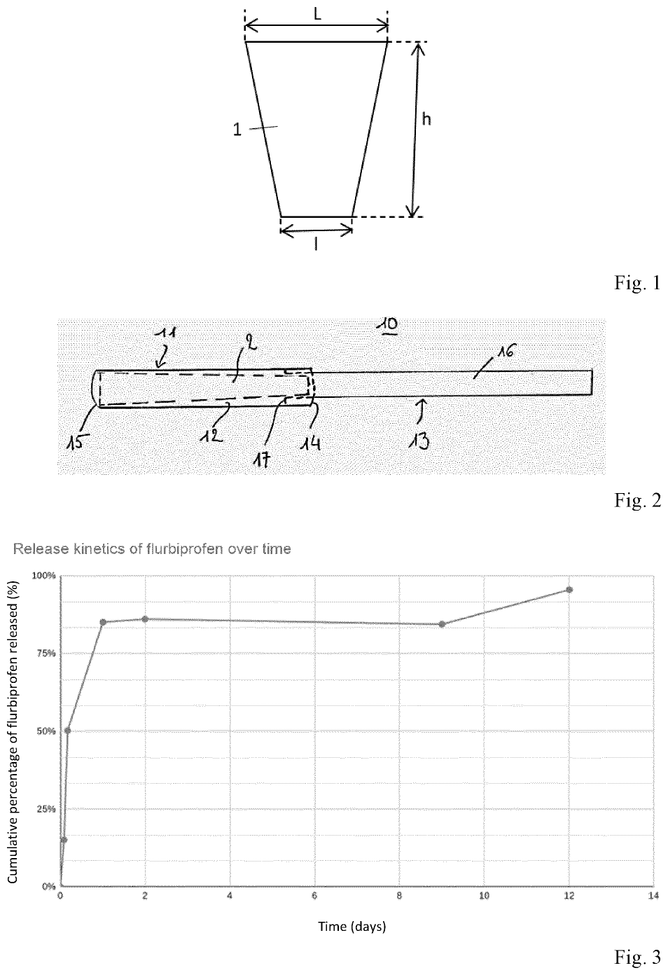

In one particular embodiment, as represented in FIG. 1, the film 1 has a trapezium shape with a height h between 1 and 4 cm approximately, of larger width L between 1 and 2.5 cm approximately, and of smaller width 1 between 0.5 and 1.5 cm approximately. For example, the trapezium has a height of around 2.5 cm, a larger width L of around 2 cm and a smaller width 1 of around 1 cm. These dimensions are easily adaptable by the person skilled in the art, notably in the above ranges, depending on the type of patient to be treated, whether she is primipara or multipara, her age, the uterine anatomy, reasons for fearing the occurrence of synechiae, etc.

The intrauterine system according to the invention is notably suitable for use in the treatment of gynaecological disorders such as fibroids, endometrial cancer, poor endometrial receptivity, thin endometrium, infections, haemorrhage (menorrhagia, metrorrhagia), cancer side effects, ageing, menopause, or vaginal dryness, or pelvic pain such as dysmenorrhoea, endometriosis, or adenomyosis in female mammals, and more particularly in women.

### The Kit

The invention also relates to a kit comprising an intrauterine system according to the invention, and to means for inserting the system in the uterine cavity. The kit according to the invention advantageously comprises means for inserting and positioning the material in the uterine cavity.

For example, as is represented in FIG. 2, the kit 10 according to the invention may comprise a hollow cylindrical inserter 11, in the bore 12 of which a film 2 having an inverted trapezium shape is housed. Advantageously, in order to minimize the dimensions of the inserter 11, the film is housed in the bore 12 in a compacted form. For example, the film is accordion folded and held closed by the internal walls of the bore 12. It is only once released in the uterine cavity that the accordion unfolds. This unfolding is furthermore promoted by the almost concomitant increase in the volume of the film, the polymers of which swell with water in contact with the intrauterine fluid.

The kit 10 advantageously comprises a plunger 13, mounted so as to slide in translation, at a distal end 14 of the inserter, the opposite proximal end 15 being the end via which the inserter 11 is intended to be introduced into the uterine cavity. The plunger 13 consists of a rod 16 which, when it is pushed inside the bore 12 of the inserter 11, towards the proximal end 15, translatably drives the film 2 out of the inserter 11.

Advantageously, the plunger 13 comprises stop means 17 at the proximal end of the rod 16, said stop means 17 being intended to come up against the wall of the inserter 11 bordering the proximal end 15 of the inserter, in order to inform the person handling the kit 10 that the film 2 has been fully ejected from the inserter and that it is in position in the uterine cavity. It is then sufficient to remove the insertion means/inserter assembly by simple pulling out, the film 2 itself remaining in position in the uterine cavity.

The kit according to the invention, and notably the insertion means, makes it possible to reliably introduce and position the degradable intrauterine system according to the invention. Furthermore, the compacted form of the intrauterine system according to the invention in dry form (before swelling) makes it possible to reduce the dimensions of the kit, which facilitates the introduction through the uterine cervix of the patient.

The kit according to the invention may notably be used in patients suffering from gynaecological disorders such as fibroids, endometrial cancer, poor endometrial receptivity, thin endometrium, infections, haemorrhage (menorrhagia, metrorrhagia), cancer side effects, ageing, menopause, or vaginal dryness, or pelvic pain such as dysmenorrhoea, endometriosis, or adenomyosis. The compacted form of the material and the use of an applicator of small dimensions facilitate the positioning thereof in the, often sensitive, uterine cavity of these patients. Moreover, the natural elimination thereof during the menstrual cycle enables the patients to avoid an additional intervention by medical personnel for removing said device.

The invention will now be illustrated using the examples below. These examples are presented by way of indication and do not in any way limit the invention.

### EXAMPLES

### Example 1: Preparation of a Degradable Intrauterine System for the Prolonged Release of Flurbiprofen in the Uterine Cavity

### 1. Synthesis of the ABA Triblock Copolymers

a. Material

Commercial poly (ethylene oxide) (PEO): Supplier Sigma Aldrich, CAS no. 25322-68-3. The commercial PEO was analysed in the laboratory by size exclusion chromatography (SEC) in order to determine its weight-average molar mass (Mw). The analysis was carried out in an analytical solvent (dimethylformamide), and the Mw was determined via a standard range of poly (ethylene glycol). The weight-average molar mass Mw is 95 000 Da and its inherent viscosity is 0.16 ml/mg.

Commercial D,L-lactide: supplier Corbion Purac, CAS no. 95-96-5.

b. Method

The ABA triblock is synthesized in the following manner:

The PEO (Mw 95,000) (200 g) and D, L-lactide (458 g) are dried under vacuum at ambient temperature for 24 h. The PEO and the D,L-lactide are introduced into a round-bottom polymerization flask in the presence of tin octanoate (85 mg). 10 successive cycles of vacuum (10⁻³ bar) and of argon are then carried out. The mixture is then heated at 140° C. and 10 successive cycles of vacuum and argon are again carried out. The mixture is returned to ambient temperature, then placed in an ice bath. Once crystallized, the reaction mixture is placed under dynamic vacuum for 30 min, then sealed under dynamic vacuum. The mixture is then placed in an oven with mechanical rotation at 140° C. for 3 days. The mixture is solubilized in dichloromethane and precipitated from an ether/ethanol mixture. The precipitate is recovered then dried under vacuum for 24 h.

c. Characterization

The ABA triblock was analysed in the laboratory by size exclusion chromatography (SEC) in order to determine its weight-average molar mass (Mw). The analysis was carried out in an analytical solvent (dimethylformamide), and the Mw was determined via a standard range of poly (ethylene glycol). The weight-average molar mass Mw is 113 000 Da. The triblock was analysed by falling ball viscometer in chloroform at 25° C., at a concentration of 0.05 g/ml. The inherent viscosity of the ABA triblock is 0.051 ml/mg.

### 2. Mixing of the ABA Triblock Copolymer, a PCL Homopolymer and Flurbiprofen

a. Material

The homopolymer added to the ABA triblock is commercial polycaprolactone (PCL), supplier Evonik Operations GmbH, CAS no. 24980-41-4, and was analysed in the laboratory by size exclusion chromatography (SEC) in order to determine its weight-average molar mass (Mw). The analysis was carried out in an analytical solvent (dimethylformamide), and the Mw was determined via a standard range of poly (methyl methacrylate) (PMMA). The weight-average molar mass Mw is 140 000 Da. The inherent viscosity is provided by the supplier. The inherent viscosity of the PCL (25° C., 0.1%, chloroform) is 1.82 dl/g.

The active ingredient is flurbiprofen, supplier Sigma Aldrich, CAS no. 5104-49-4.

b. Method

80% by weight of the ABA triblock (3.2 g) and 20% by weight of the PCL homopolymer (0.8 g) are solubilized in 40 ml of dichloromethane with stirring at ambient temperature. The flurbiprofen (0.4 g, equivalent to 10% of the total weight of polymer, i.e. 9% by weight of the total weight of the system) is added to the mixture and the mixture is stirred for 4 h. The mixture is dried on a rotary evaporator under vacuum at 100 mbar at temperature until the dichloromethane is eliminated. The dry mixture is recovered.

### 3. Preparation of the Flurbiprofen-Releasing Intrauterine System According to the Invention

a. Forming

The mixture comprising the ABA triblock, the homopolymer and the flurbiprofen is formed by hot pressing. The mixture is pressed between two heated platens at 85° C. for 8 min to which a pressure of 20 mPa is applied between the two platens. The thickness of the film depends on the amount of polymer used and on the surface area of the support. The polymer film obtained has a thickness of 500 microns. The film is then cut using a punch in the shape of a trapezium with a height h of 2.5 cm, largest width L of 2 cm and smallest width 1 of 1 cm. The film has a weight of around 200 mg, including 9% flurbiprofen, i.e. 18 mg of flurbiprofen.

### 4. Evaluation of the Properties of the Intrauterine System According to the Invention

a. Methods
i. The in vitro Conditions of the Release Study

The trapezoidal film is deposited in a sealed 50-ml glass flask containing 33 ml of phosphate-buffered saline (pH 7.4). The flasks (n=3) are placed at 37° C. under mechanical stirring (100 rpm). 1 ml of solution is sampled after 2 h, 4 h, 1 day, 2 days, 9 days and 12 days of release. After each sampling, 1 ml of phosphate-buffered saline is added to the medium, except for the sampling on the 9th day where the entire medium is replaced with 33 ml of phosphate-buffered saline. Each sample, i.e. n=3 samples per time, is analysed by high performance liquid chromatography (HPLC). The amount of flurbiprofen released is calculated for each sample time from the equation of the calibration curve.

The concentration of flurbiprofen (μg/ml) released at time t is calculated as follows:

$$Concentration of flubiprofen ( t ) ( µg / ml ) = Area under the curve measured in HPLC / a , with a being the value of the equation of the calibration curve y = ax .$$

The amount of flurbiprofen (μg) released in the medium at time t is calculated as follows:

$$Amount of flurbiprofen ( t ) ( μ g ) = Total volume ( ml ) of medium \times Concentration of flurbiprofen ( t ) \\ ( μ g / ml )$$

The cumulative percentage of flurbiprofen released at time t is then calculated in the following manner:

$$Cumulative percentage of flurbiprofen ( t ) ( \% ) = ( Amount of flurbiprofen ( t ) ( μ g ) / Initial amount of flurbiprofen ( μ g ) ) \times 100 \%$$

ii. Method for Measuring the Increase in Surface Area of the Film

The increase in surface area of the film is measured after 24 h of release under in vitro conditions. The film is removed and the surface area of the film is measured using the ImageJ software. The increase in surface area is measured as follows:

$$Increase in surface area ( \% ) = ( ( Surface area ( t = 24 h ) - Surface area ( t = 0 ) ) / Surface area ( t = 0 ) ) \times 100$$

iii. Evaluation of the Degradation of the Film Under in Vitro Degradation Conditions

The trapezoidal film is deposited in a sealed 50-ml glass flask containing 33 ml of phosphate-buffered saline (pH 7.4). The flasks (n=3) are placed at 37° C. under mechanical stirring (100 rpm). The appearance of the films is evaluated after 24 h, 15 days and 70 days of in vitro degradation. After 15days of degradation, the films are dried with a freeze dryer at −60° C. under 0.025 mbar for 24 h to obtain a stable mass. The dry films are weighed to determine the loss of mass of the films after 15 days of degradation. The loss of mass of the films is calculated as follows:

$$Loss of mass ( \% ) = ( ( Mass ( t ) - Mass ( t = 0 ) ) / Mass ( t = 0 ) ) \times 100$$

iv. Methods for Quantifying the Release of Flurbiprofen by High Performance Liquid Chromatography (HPLC)

The separation by high performance liquid chromatography (HPLC) is carried out using an HPLC system (Shimadzu) comprising a Kinetex C18 column (2.6 μm; 100×4.6 mm, Phenomenex, California, USA) maintained at 30° C. The mobile phase is composed of 60% water and 40% acetonitrile (60/40 v/v). The isocratic flow rate is 1.0 ml/min. The injection volume is 10 μl. The detection is carried out at a wavelength of 247 nm.

The calibration curve is produced by solubilizing 1 mg of pure active ingredient in 40 ml of phosphate-buffered saline (PBS) in a sealed 50-ml glass flask (n=3). From this stock solution, a serial dilution is carried out using PBS to obtain a concentration range of from 2.5 to 25 μg/ml.

b. Results
i. Increase in the Surface Area of the Film

After 24 h of release, the film has a surface area of 808 mm2, i.e. an increase in surface area of 115% relative to the initial surface area of the film.

ii. Release Kinetics

### Calibration Curve

The calibration curve is linear (r2=0.99968) over the concentration range of 2.5 to 25 μg/ml.

### Release Kinetics

FIG. 3 shows the cumulative percentage of flurbiprofen released over time from the film composed of the ABA triblock and PCL homopolymer mixture. Under in vitro release conditions, flurbiprofen is released from the polymer film over time, with a burst effect over the 1st day, then a release up to the 12th day. The intrauterine system enables a release of flurbiprofen over 12 days under in vitro release conditions.

iii. Degradation

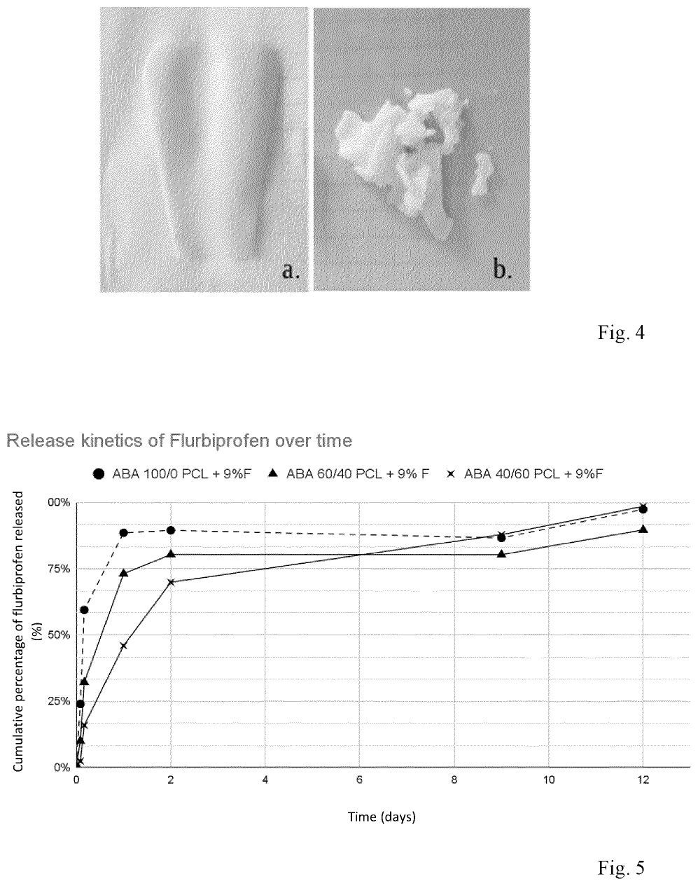

FIG. 4 shows the appearance of the films composed of the ABA triblock and PCL homopolymer mixture after 24 h of degradation (FIG. 4.a) and 15 days of degradation (FIG. 4.b) under in vitro degradation conditions. The film is a malleable and elastic barrier after 24 h of degradation. After 15 days of degradation, the film loses its mechanical properties since the film disintegrates into several pieces when it is handled. After 15 days of in vitro degradation, the film has lost 30% of its initial mass, and can be easily evacuated by natural routes under the clinical usage conditions. After 70 days of in vitro degradation, the film is completely solubilized in the degradation medium.

### Example 2: Preparation of a Degradable Intrauterine System for the Sustained Release of Flurbiprofen in the Uterine Cavity

### 1—Synthesis of ABA Triblock Copolymers

Same as Example 1, Section 1 Synthesis of ABA triblock copolymers

### 2—Mix of ABA Triblock Copolymer, PCL Homopolymer and Flurbiprofen

a. Material

Same as Example 1, Section 2a.

b. Methods

60 wt % of the triblock ABA (2.4 g) and 40 wt % of the homopolymer PCL (1.6 g) are solubilized in 40 ml of dichloromethane under stirring at room temperature. Flurbiprofen (0.4 g, equivalent to 9% by mass, based on the total mass of the system) is added to the mixture and the mixture is stirred for 4 H. The mixture is dried in a rotavapor under vacuum at 100 mbar at temperature until the dichloromethane is removed. The dry mix is recovered. The formulation is named “ABA 60/40 PCL+9% F”, with “F” the Flurbiprofen.

40 wt % of the triblock ABA (1.6 g) and 60 wt % of the homopolymer PCL (2.4 g) are solubilized in 40 ml of dichloromethane under stirring at room temperature. Flurbiprofen (0.4 g, equivalent to 9% by mass, based on the total mass of the system) is added to the mixture and the mixture is stirred for 4 H. The mixture is dried in a rotavapor under vacuum at 100 mbar at temperature until the dichloromethane is removed. The formulation is named “ABA 40/60 PCL+9% F”, with “F” the Flurbiprofen.

100 wt % of the triblock ABA (4 g) and 0 wt % of the homopolymer PCL (0 g) are solubilized in 40 ml of dichloromethane under stirring at room temperature. Flurbiprofen (0.4 g, equivalent to 9% by mass, based on the total mass of the system) is added to the mixture and the mixture is stirred for 4 H. The mixture is dried in a rotavapor under vacuum at 100 mbar at temperature until the dichloromethane is removed. The formulation is named “ABA 100/0 PCL+9% F”, with “F” the Flurbiprofen.

### 3—Preparation of the Intrauterine Delivery System of Flurbiprofen According to the Invention

a. Shaping Process

The mixture comprising triblock ABA, optionally homopolymer and flurbiprofen is shaped by hot pressing. The mixture is pressed between two heating platens at 85° C. for 8 min and a pressure of 20 mPa is applied between the two platens. The thickness of the film depends on the amount of polymer used and the surface of the substrate. The resulting polymer films have a thickness of 500 microns. The film is then cut with a punch in the form of a trapezoid with a height h of 2.5 cm, the largest width L of 2 cm and the smallest width 1 of 1 cm. The film has a mass of about 200 mg of which 9% is Flurbiprofen, i.e. 18 mg of Flurbiprofen.

### 4—Evaluation of the Properties of the Intrauterine System According to the Invention

a. Methods
i. In-vitro Conditions of the Release Study

The trapezoidal film is placed in a sealed 50 ml glass vial containing 33 ml of phosphate buffer (pH 7.4). The vials (n=3) are placed at 37° C. under mechanical shaking (100 rpm). 1 ml of solution is collected after 2 H, 4 H, 1 day, 2 days, 9 days and 12 days of release. After each sampling, 1 ml of saline phosphate buffer is added to the medium, except for the sample at day 9 where the whole medium is replaced by 33 ml of saline phosphate buffer. Each sample, i.e. n=3 samples per time, are analyzed by High Performance Liquid Chromatography (HPLC). The amount of flurbiprofen released is calculated for each sample time from the equation of the calibration curve.

The concentration of Flurbiprofen (μg/ml) released at time t is calculated as follows:

$$Flurbiprofen concentration ( t ) ( μ g / ml ) = Area under curve measured in HPLC / a , with a the value of the equation of the calibration curve y = ax .$$

The amount of Flurbiprofen (μg) released into the medium at time t is calculated as follows:

$$Amount of Flurbiprofen ( t ) ( μ g ) = Total volume ( ml ) of medium \times Concentration of Flurbiprofen ( t ) ( μ g / ml )$$

The cumulative percentage of Flurbiprofen released at time t is then calculated as follows:

$$Cumulative percentage of Flurbiprofen ( t ) ( \% ) = ( Amount of Flurbiprofen ( t ) ( μ g ) / Initial amount of Flurbiprofen ( μ g ) ) \times 100 \%$$

ii. Methods for Quantification of Flurbiprofen Release by High Performance Liquid Chromatography (HPLC)

The quantification by High Performance Liquid Chromatography (HPLC) is performed using a HPLC system (Shimadzu) comprising a Kinetex C18 column (2.6 μm; 100×4.6 mm, Phenomenex, California, USA) maintained at 30° C. The mobile phase is composed of 60% water and 40% Acetonitrile (60/40 v/v). The isocratic flow rate is 1.0 ml/min. The injection volume is 10 μl. The detection is performed at a wavelength of 247 nm.

The calibration curve is performed by solubilizing 1 mg of pure active ingredient in 40 ml of phosphate- buffered saline (PBS) in a sealed 50 ml glass vial (n=3). From this stock solution, a series of dilutions is performed using PBS to obtain a concentration range from 2.5 to 25 μg/ml.

b. Results
i. Release Kinetics

### Calibration Curve

The calibration curve is linear (r2=0.99968) over the concentration range of 2.5 to 25 μg/ml.

### Release Kinetics

FIG. 5 shows the cumulative percentage of flurbiprofen released over time from ABA 60/40 PCL and ABA 40/60 PCL films composed of the triblock ABA mixture and the homopolymer PCL, and ABA 100/0 PCL films composed of the triblock ABA alone. Under in-vitro release conditions, flurbiprofen releases from the polymer film over time, with a burst effect during day 1 and then a release until day 12. The addition of PCL to the mixture modulates the release kinetics of flurbiprofen during the first days. The intrauterine system allows a release of flurbiprofen for 12 days under in-vitro release conditions. In absence of PCL homopolymer, more than 50% of the flurbiprofen is released in 4 hours and this formulation is not acceptable for a prolonged-release.

### Exemple 3: Preparation of a Degradable Intrauterine System for the Sustained Release of Flurbiprofen in the Uterine Cavity

### 1—Synthesis of ABA Triblock Copolymers

Same as Example 1, Section 1 Synthesis of ABA triblock copolymers

### 2—Mixture of ABA Triblock Copolymer, PCL Homopolymer and Flurbiprofen

a. Material

Same as Example 1, Section 2a.

b. Methods

30 wt % of ABA triblock (96 mg), 19 wt % of PCL homopolymer (64 mg), 51 wt % of PLA50 homopolymer (166.4 mg) are solubilized in 40 ml of dichloromethane under stirring at room temperature. Flurbiprofen (90 mg, equivalent to 22% by mass, based on the total mass of the system) is added to the mixture and the mixture is stirred for 4H. The mixture is rotavapor dried under vacuum at 100 mbar at temperature until the dichloromethane is removed. The formulation is named “ABA 30/70 PCL/PLA50+22% F”, with “F” the Flurbiprofen.

20 wt % of ABA triblock (64 mg), 29 wt % of PCL homopolymer (96 mg), 51 wt % of PLA50 homopolymer (166.4 mg) are solubilized in 40 ml of dichloromethane under stirring at room temperature. Flurbiprofen (90 mg, equivalent to 22% by mass, based on the total mass of the system) is added to the mixture and the mixture is stirred for 4H. The mixture is rotavapor dried under vacuum at 100 mbar at temperature until the dichloromethane is removed. The formulation is named “ABA 20/80 PCL/PLA50+22% F”, with “F” the Flurbiprofen.

### 3—Preparation of the Intrauterine Delivery System of Flurbiprofen According to the Invention

a. Shaping Process

The mixture comprising triblock ABA, homopolymer PCL, homopolymer PLA50 and flurbiprofen is shaped by hot pressing. The mixture is pressed between two heating platens at 85° C. for 8 min and a pressure of 20 mPa is applied between the two platens. The thickness of the film depends on the amount of polymer used and the surface of the substrate. The resulting polymer films have a thickness of 1000 microns. The film is then cut with a punch in the form of a trapezoid with a height h of 2.5 cm, the largest width L of 2 cm and the smallest width 1 of 1 cm. The film has a mass of about 416 mg of which 22% is Flurbiprofen, i.e. about 90 mg of Flurbiprofen.

### 4—Evaluation of the Properties of the Intrauterine System According to the Invention

c. Methods
i. In-vitro Conditions of the Release Study

A 50 mg sample of the trapezoidal film is cut and placed in a sealed 50 ml glass vial containing 45 ml of saline phosphate buffer (pH 7.4). The vials (n=3) are placed at 37° C. under mechanical agitation (100 rpm). 1 ml of solution is collected after 2 H, 4 H, 1 day, 2 days, 7 days and every 7 days until 56 days of release. After each sampling, 1 ml of phosphate buffer is added to the medium. Each sample, i.e. n=3 samples per time, are analyzed by High Performance Liquid Chromatography (HPLC). The amount of flurbiprofen released is calculated for each sampling time from the equation of the calibration curve.

The concentration of Flurbiprofen (μg/ml) released at time t is calculated as follows:

$$Flurbiprofen concentration ( t ) ( μ g / ml ) = Area under curve measured in HPLC / a , with a the value of the calibration curve equation y = ax .$$

The amount of Flurbiprofen (μg) released into the medium at time t is calculated as follows:

$$Amount of Flurbiprofen ( t ) ( μ g ) = Total volume ( ml ) of medium \times Concentration of Flurbiprofen ( t ) ( μ g / ml )$$

The cumulative percentage of Flurbiprofen released at time t is then calculated as follows:

$$Cumulative percentage of Flurbiprofen ( t ) ( \% ) = ( Amount of Flurbiprofen ( t ) ( μ g ) / Initial amount of Flurbiprofen ( μ g ) ) \times 100 \%$$

ii. Methods for Quantification of Flurbiprofen Release by High Performance Liquid Chromatography (HPLC)

The quantification by High Performance Liquid Chromatography (HPLC) is performed using a HPLC system (Shimadzu) comprising a Kinetex C18 column (2.6 μm; 100×4.6 mm, Phenomenex, California, USA) maintained at 30° C. The mobile phase is composed of 60% water and 40% Acetonitrile (60/40 v/v). The isocratic flow rate is 1.0 ml/min. The injection volume is 10 μl. The detection is performed at a wavelength of 247 nm.

The calibration curve is performed by solubilizing 1 mg of pure active ingredient in 40 ml of phosphate-buffered saline (PBS) in a sealed 50 ml glass vial (n=3). From this stock solution, a series of dilutions is performed using PBS to obtain a concentration range from 2.5 to 25 μg/ml.

d. Results
i. Release Kinetics

### Calibration Curve

The calibration curve is linear (r2=0.99968) over the concentration range of 2.5 to 25 μg/ml.

### Release Kinetics

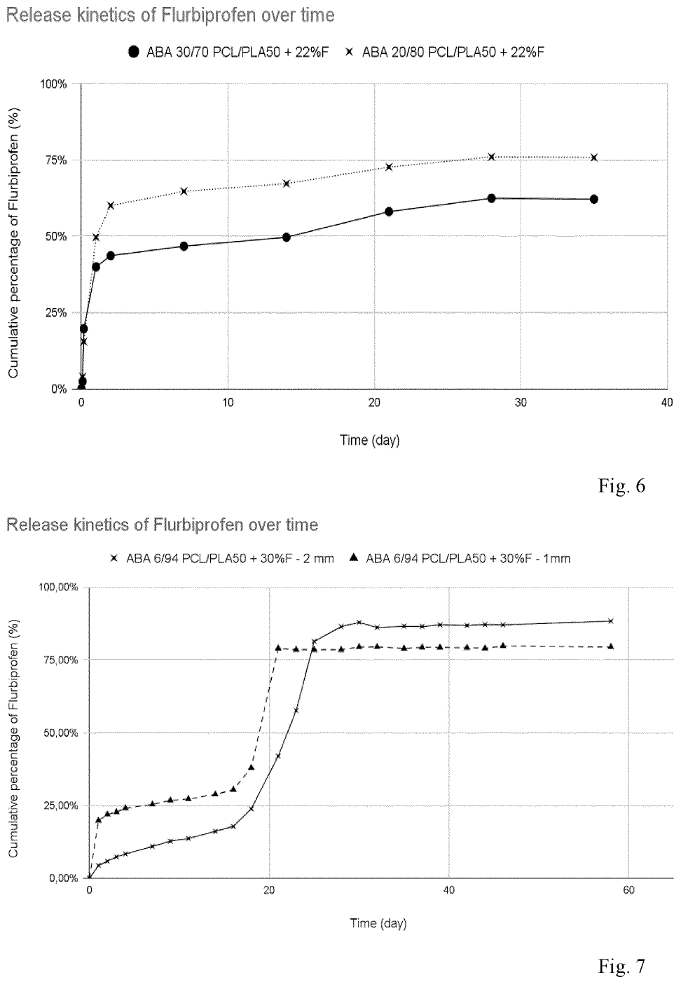

FIG. 6 shows the cumulative percentage of flurbiprofen released over time from ABA 30/70 PCL/PLA50 and ABA 20/80 PCL/PLA50 films, composed of the triblock mixture ABA and the homopolymer PCL and PLA50. Under in-vitro release conditions, flurbiprofen releases from the polymer film over time, with a burst effect during day 1 and then a release until day 35. The addition of tribloc ABA to the mixture modulates the release kinetics of flurbiprofen over time. The intrauterine system allows a release of flurbiprofen for 35 days under in-vitro release conditions. The increase of the percentage of PLA in the homopolymer fraction of the system delays the release of flurbiprofen.

### Example 4: Preparation of a Degradable Intrauterine System for the Sustained Release of Flurbiprofen in the Uterine Cavity

### 1—Synthesis of ABA Triblock Copolymers

Same as Example 1, Section 1 Synthesis of ABA triblock copolymers

### 2—Mixture of ABA Triblock Copolymer, PCL Homopolymer and Flurbiprofen

a. Material

### 2—Same as Example 1, Section 2a.

a. Methods

20 wt % of ABA triblock (66.56 mg), 30 wt % of PCL homopolymer (99.84 mg), 50 wt % of PLA50 homopolymer (166.38 mg) are solubilized in 40 ml of dichloromethane under stirring at room temperature. Flurbiprofen (83.19 mg, equivalent to 20% by mass, based on the total mass of the system) is added to the mixture and the mixture is stirred for 4 H. The mixture is dried in a rotavapor under vacuum at 100 mbar at temperature until the dichloromethane is removed. The formulation is named “ABA 20/80 PCL/PLA50+20% F”, with “F” the Flurbiprofen.

6 wt % of ABA triblock (52 mg), 9 wt % of PCL homopolymer (78.67 mg), 85 wt % of PLA50 homopolymer (748.3 mg) are solubilized in 40 ml of dichloromethane under stirring at room temperature. Flurbiprofen (374.83 mg, equivalent to 30% by mass, based on the total mass of the system) is added to the mixture and the mixture is stirred for 4 H. The mixture is rotavapor dried under vacuum at 100 mbar at temperature until the dichloromethane is removed. The formulation is named “ABA 6/94 PCL/PLA50+30% F”, with “F” the Flurbiprofen.

### 3—Preparation of the Intrauterine Delivery System of Flurbiprofen According to the Invention

a. Shaping Process

The mixture comprising triblock ABA, homopolymer PCL, homopolymer PLA50 and flurbiprofen is shaped by hot pressing. The mixture is pressed between two heating platens at 85° C. for 8 min and a pressure of 20 mPa is applied between the two platens. The thickness of the film depends on the amount of polymer used and the surface of the substrate. The resulting polymer films have a thickness of 1000 microns for “ABA 20/80 PCL/PLA50+20% F—1 mm” and “ABA 6/94 PCL/PLA50+30% F-1 mm”, and a thickness of 2000 microns for “ABA 6/94 PCL/PLA50+30% F-2 mm”. The film is then cut into a trapezoidal shape with a height h of 2.5 cm, the largest width L of 2 cm and the smallest width 1 of 1 cm. The films “ABA 20/80 PCL/PLA50+20% F-1 mm” and “ABA 6/94 PCL/PLA50+30% F-1 mm” have a mass of about 416 mg with 20% Flurbiprofen, i.e. about 83 mg Flurbiprofen, and 30% Flurbiprofen, i.e. about 124.8 mg Flurbiprofen, respectively. The film “ABA 6/94 PCL/PLA50+30% F-2 mm” have a mass of about 832 mg with 30% Flurbiprofen, i.e. about 250 mg of Flurbiprofen.

### 4—Evaluation of the Properties of the Intrauterine System According to the Invention

e. Methods
i. In-vitro Conditions of the Release Study

Conditions for “ABA 20/80 PCL/PLA50+20% F-1 mm” and “ABA 6/94 PCL/PLA50+30% F-1 mm” formulations: a 14 mg sample of the trapezoidal film is cut and placed in a 50 ml sealed glass vial containing 14 ml of saline phosphate buffer (pH 7.4)

Conditions for the “ABA 6/94 PCL/PLA50+30% F-2 mm” formulation: a 28 mg sample of the trapezoidal film is cut and placed in a sealed 50 ml glass vial containing 28 ml of phosphate buffer (pH 7.4).

The vials (n=3) are placed at 37° C. under mechanical agitation (100 rpm). 1 ml of solution is taken after each day, or at intervals of 3 days, until 56 days of release. After each sampling, 1 ml of phosphate saline buffer is added to the medium. Each sample, i.e. n=3 samples per time, are analyzed by High Performance Liquid Chromatography (HPLC). The amount of flurbiprofen released is calculated for each sampling time from the equation of the calibration curve.

The concentration of Flurbiprofen (μg/ml) released at time t is calculated as follows:

$$Flurbiprofen concentration ( t ) ( μ g / ml ) = Area under curve measured in HPLC / a , with a the value of the calibration curve equation y = ax .$$

The amount of Flurbiprofen (μg) released into the medium at time t is calculated as follows:

$$Amount of Flurbiprofen ( t ) ( μ g ) = Total volume ( ml ) of medium \times Concentration of Flurbiprofen ( t ) ( μ g / ml )$$

The cumulative percentage of Flurbiprofen released at time t is then calculated as follows:

$$Cumulative percentage of Flurbiprofen ( t ) ( \% ) = ( Amount of Flurbiprofen ( t ) ( μ g ) / Initial amount of Flurbiprofen ( μ g ) ) \times 100 \%$$

ii. Methods for Quantification of Flurbiprofen Release by High Performance Liquid Chromatography (HPLC)

The quantification by High Performance Liquid Chromatography (HPLC) is performed using a HPLC system (Shimadzu) comprising a Kinetex C18 column (2.6 μm; 100×4.6 mm, Phenomenex, California, USA) maintained at 30° C. The mobile phase is composed of 60% water and 40% Acetonitrile (60/40 v/v). The isocratic flow rate is 1.0 ml/min. The injection volume is 10 μl. The detection is performed at a wavelength of 247 nm.

The calibration curve is performed by solubilizing 10 mg of pure active ingredient in 10 ml of phosphate-buffered saline (PBS) in a sealed 50 ml glass vial (n=3). From this stock solution, a series of dilutions is performed using PBS to obtain a concentration range from 25 to 1000 μg/ml.

iii. In Vivo Study

A 1 mm thick film, “ABA 20/80 PCL/PLA50+20% F”, was implanted in the uterus of female rats (Sprague Dawley, 8 weeks) in order to evaluate:
The percentage of water intake,
The loss of mass over time,
The amount of flurbiprofen absorbed into the uterine tissues.

Flurbiprofen was administered orally to another group of female rats to assess the amount of flurbiprofen absorbed into the uterine tissues and serve as a control arm.

### Surgical Procedure

### Procedure for Film Implantation

The formulation was shaped according to Section 3.a. A sample of approximately 15 mg of the trapezoidal film is cut and implanted into the uterus (also known as the uterine horn) of the rat after anesthesia with 2% Isoflurane, laparotomy and incision of the uterine horn. Each rat has two rat uterine horns. A specimen is therefore implanted in each uterine horn. The horn is then sutured and a ligature is made on the vaginal side to avoid vaginal evacuation of the device.

### Procedure for Oral Administration

4 mg/kg of Flurbiprofen was administered orally twice daily. Flurbiprofen was first solubilized in a 0.5 mg/ml Hydroxyethyl cellulose solution (Cellosize).

Rats received 300 mg/kg paracetamol 24 hours before surgery, and 0.05 mg/kg Buprenorphine subcutaneously after anesthesia and before the start of surgery.

At each sacrifice time, the uterine horns were incised lengthwise and film samples were taken. The groups are divided as follows (see Table 1):

TABLE 1

| | | Number of rats/ | |
|---|---|---|---|
| | | Number of uterine | Time of |
| Group | Formulation | horns | sacrifice |
| 1 | Flurbiprofen by oral | 3 rats | +11 H |
| | administration | | |
| 2A | ABA 20/80 PCL/PLA50 + | 3 rats/6 uterine | +11 H |
| | 20% F | horns | |
| 3 | Flurbiprofen by oral | 3 rats | DAY 1 |
| | administration | | |
| 4A | ABA 20/80 PCL/PLA50 + | 3 rats/6 uterine | DAY 1 |
| | 20% F | horns | |
| 5 | Flurbiprofen by oral | 3 rats | DAY 15 |
| | administration | | |
| 6A | ABA 20/80 PCL/PLA50 + | 3 rats/6 uterine | DAY 15 |
| | 20% F | horns | |
| 7A | ABA 20/80 PCL/PLA50 + | 3 rats/6 uterine | DAY 28 |
| | 20% F | horns | |
| 8A | ABA 20/80 PCL/PLA50 + | 3 rats/6 uterine | DAY 56 |
| | 20% F | horns | |

Method for Measuring the Water Absorption of the Film Under in-vivo Conditions

The water uptake of the film is measured 11 hours after implantation in the uterine horns (group 2A). The film is placed on absorbent paper and weighed. The percentage of water absorption is measured as follows:

$$Water intake ( \% ) = ( ( Mass ( t = 11 h ) - Mass ( t = 0 ) ) / Mass ( t = 0 ) ) \times 100$$

Evaluation of Film Degradation Under in-vivo Degradation Conditions

At each sacrifice time, the film is taken and then dried by freeze-drying at −60° C. under 0.025 mbar for 24 H to obtain a stable mass. The dried films are weighed to determine the mass loss of the films. The mass loss of the films is calculated as follows:

$$Mass loss ( \% ) = ( ( Mass ( t ) - Mass ( t = 0 ) ) / Mass ( t = 0 ) \times 100$$

Evaluation of the Amount of Flurbiprofen Absorbed in Uterine Tissue Under in-vivo Conditions

At each sacrifice time, uterine horns were collected and analyzed by Liquid Chromatography (LC30AD mounted on an Agilent C18 column) coupled with Mass Spectrometry (Shimadzu TripleQuad 8060) (LC-MS).

The uterine horn is placed in an eppendorf and ground with 0.5 mL of 1X PBS. 100 μL of the homogenate is collected. 20 μL of 1 mg/mL Deuterium (D5) labeled flurbiprofen is added to the homogenate. 430 μL of methanol: water mixture (ratio 8:1, −20° C.) is then added, mixed by inversion and stirred at 4° C. for 20 minutes. The mixture is centrifuged for 5 minutes at 16,000 g at 4° C. The mixture is placed on a Captiva EMR-Lipids plate, conditioned with 200 μL of 0.1% ACN Formic Acid (FA). The plate is centrifuged for 2 minutes at 1000 rpm. 400 μL of supernatant is transferred to the Captiva EMR-Lipids plate. The plate is centrifuged for 45 minutes at 1000 rpm, dried for 3 hours at 30° C. 60 μL of methanol is added and 20 μL is injected. The samples were analysed in positive mode.

a. Results
i. Release Kinetics

### Calibration Curve

The calibration curve is linear (r2=0.99968) over the concentration range of 2.5 to 25 μg/ml.

### Release Kinetics

FIG. 7 shows the cumulative percentage of flurbiprofen released over time from the 1,000 μm and 2,000 μm thick ABA 6/94 PCL/PLA50+30% F films, composed of the triblock mixture ABA and the homopolymer PCL and PLA50. Under in-vitro release conditions, flurbiprofen releases from the polymer film over time. The thickness of the film modulates the release kinetics of flurbiprofen.

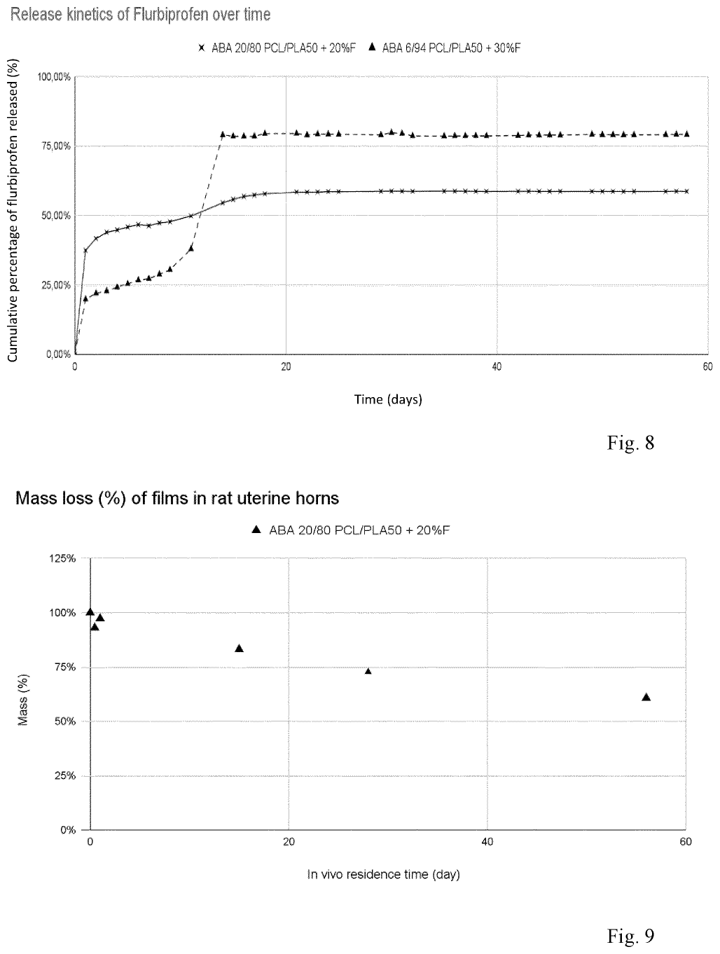

FIG. 8 shows the cumulative percentage of flurbiprofen released over time from the 1 mm thick films, ABA 20/80 PCL/PLA50+20% F and ABA 6/94 PCL/PLA50+30% F, composed of the triblock mixture ABA and the homopolymer PCL and PLA50. Under in-vitro release conditions, flurbiprofen releases from the polymer film over time, with a burst effect during day 1, followed by release until day 22 for ABA 20/80 PCL/PLA50+20% F and until day 14 for ABA 6/94 PCL/PLA50+30% F.

ii. Measurement of Film Water Absorption Under in-vivo Conditions

An average water uptake of 65.34% (Sd 3,49%) is observed for the “ABA 20/80 PCL/PLA50+20% F” films.

iii. Measurement of Film Mass Loss Under in-vivo Degradation Conditions

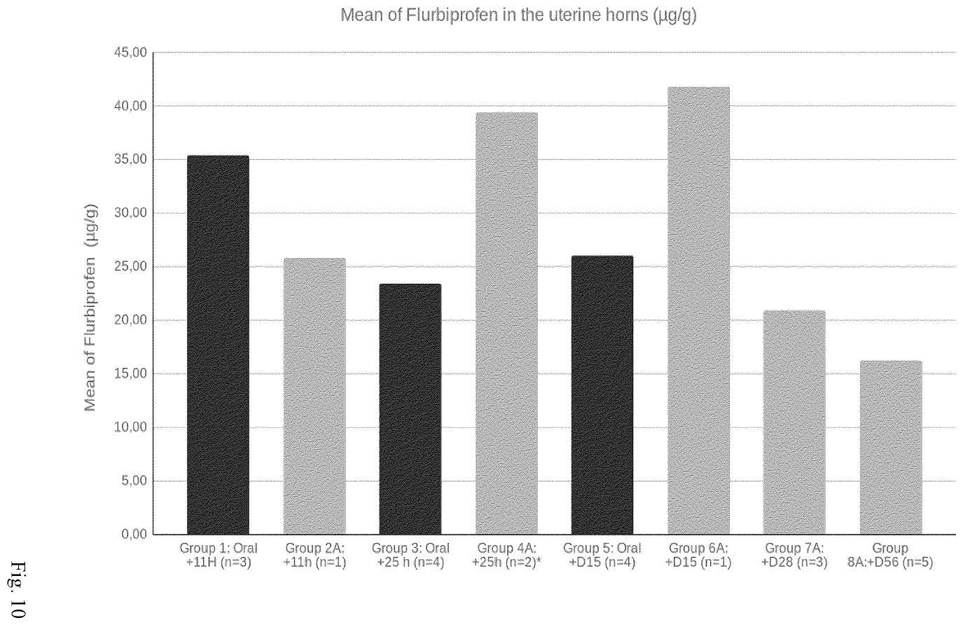

FIG. 9 shows the mass loss of ABA 20/80 PCL/PLA50+20% F films at different times after being implanted into rat uterine horns. The “ABA 20/80 PCL/PLA50+20% F” films lost 40% of their mass at 56 days post-implantation.

iv. Measurement of the Amount of Flurbiprofen Absorbed in Uterine Tissue Under in-vivo Conditions

FIG. 10 shows the amount of flurbiprofen absorbed in rat uterine tissue after implantation of “ABA 6/94 PCL/PLA50+30% F” film and the amount of flurbiprofen absorbed in uterine tissue after oral administration of flurbiprofen. At 11 H, 25 H and 15 days after oral administration or introduction of the intrauterine release system into the horns, equivalent tissue concentrations of flurbiprofen (24-39 μg/g) are measured suggesting that the intrauterine release system may achieve the same therapeutic effectiveness. It is also important to note that the “ABA 6/94 PCL/PLA50+30% F” film releases flurbiprofen for 56 days under in vivo conditions.

1-15. (canceled)

16. A degradable intrauterine system for the prolonged release of an active ingredient in the uterine cavity comprising:
(a) a degradable A and B block copolymer, wherein:
the A block is a polyester;
the B block is a poly(oxyethylene) (PEO) with a weight-average molecular weight of greater than or equal to 50 kDa; and
the ethylene oxide unit/ester unit molar ratio is between 0.05 and 5;
(b) at least one polyester homopolymer; and
(c) at least one active ingredient for release in the uterine cavity.

17. The degradable intrauterine system according to claim 16, wherein the copolymer (a)/homopolymer (b) weight ratio is between 99/1 and 1/99.

18. The degradable intrauterine system according to claim 17, wherein the copolymer (a)/homopolymer (b) weight ratio is between 95/5 and 5/95.

19. The degradable intrauterine system according to claim 16, wherein the A and B block copolymer is selected from AB diblock copolymers and ABA and BAB triblock copolymers, and mixtures thereof.

20. The degradable intrauterine system according to claim 19, wherein the A and B block copolymer is selected from ABA and BAB triblock copolymers, and mixtures thereof.

21. The degradable intrauterine system according to claim 16, wherein the weight-average molecular weight of the B blocks in the A and B block copolymer is between 75 kDa and 150 kDa.

22. The degradable intrauterine system according to claim 21, wherein the weight-average molecular weight of the B blocks in the A and B block copolymer is between 80 and 125 kDa.

23. The degradable intrauterine system according to claim 16, wherein the ethylene oxide unit/ester unit ratio of the A and B block copolymer is from 0.1 to 4.

24. The degradable intrauterine system according to claim 23, wherein the ethylene oxide unit/ester unit ratio of the A and B block copolymer is from 0.1 to 3.

25. The degradable intrauterine system according to claim 16, wherein the A block is selected from poly(lactic acid) (PLA), poly(glycolic acid) (PGA), poly(lactic-co-glycolic acid) (PLGA), polycaprolactone (PCL), polybutyrolactone (PBL), polyhydroxyalkanoates (PHA), and copolymers thereof.

26. The degradable intrauterine system according to claim 16, wherein the A block is a polycaprolactone (PCL) or a poly(lactic acid) (PLA) comprising at least 50% of L-lactic acid.

27. The degradable intrauterine system according to claim 16, wherein the homopolymer (b) is selected from poly(lactic acid) (PLA), poly glycolic acid) (PGA), poly(lactic-co-glycolic acid) (PLGA), polycaprolactone (PCL), polybutyrolactone (PBL) and polyhydroxyalkanoates (PHA), and mixtures thereof.

28. The degradable intrauterine system according to claim 27, wherein the homopolymer (b) is a PLA and/or PCL.

29. The degradable intrauterine system according to claim 16, wherein the homopolymer (b) has a number-average molar mass of between 25,000 g/mol and 250,000 g/mol.

30. The degradable intrauterine system according to claim 16, wherein the active ingredient for release in the uterine cavity is selected from antibiotics, antifungals or antivirals; steroidal or non-steroidal anti-inflammatory drugs; vasoconstrictors; vasodilators;
uterine relaxants; oxytocics; hormones, hormone analogues, hormone agonists, hormone antagonists; anti-cancer drugs; or mixtures thereof.

31. The degradable intrauterine system according claim 16, wherein the content of active ingredient is between 0.01% and 60% by weight.

32. The degradable intrauterine system according to claim 16, wherein the active ingredient is not covalently bound to the copolymer a) or to the homopolymer b).

33. The degradable intrauterine according to claim 16, said system releasing the active ingredient over at least 10 days.

34. The degradable intrauterine system according to claim 16, said system degrading after a residence time in an aqueous or humid environment of between 10 days and 1 2months.

35. A kit comprising at least one intrauterine system according to claim 16 and means for inserting the system into the uterine cavity.
